(19) United States
(12) Patent Application Publication
(10) Pub. No.: US 2020/0281890 A1
(43) Pub. Date: Sep. 10, 2020

(54) COMPOSITIONS COMPRISING CANNABIDIOL, TETRAHYDROCANNABINOL, TERPENES, AND FLAVONOIDS AND USE THEREOF IN THE TREATMENT OF INSOMNIA

(71) Applicant: CANOPY HEALTH INNOVATIONS, Toronto (CA)

(72) Inventors: Laura MACNAIR, Toronto (CA); Rami BATAL, Toronto (CA); Mark Andrew HETHERINGTON, Toronto (CA); Mark WARE, Toronto (CA); Chris SCHNARR, Toronto (CA); Steve KRAWCYZK, Toronto (CA)

(21) Appl. No.: 16/650,525

(22) PCT Filed: Sep. 25, 2018
(86) PCT No.: PCT/CA2018/051200

§ 371 Date: Mar. 25, 2020

Related U.S. Application Data
(60) Provisional application No. 62/562,817, filed on Sep. 25, 2017.
(60) Provisional application No. 62/562,823, filed on Sep. 25, 2017.
(60) Provisional application No. 62/562,826, filed on Sep. 25, 2017.
(60) Provisional application No. 62/562,832, filed on Sep. 25, 2017.
(60) Provisional application No. 62/562,836, filed on Sep. 25, 2017.
(60) Provisional application No. 62/562,840, filed on Sep. 25, 2017.
(60) Provisional application No. 62/562,845, filed on Sep. 25, 2017.
(60) Provisional application No. 62/562,850, filed on Sep. 25, 2017.
(60) Provisional application No. 62/562,853, filed on Sep. 25, 2017.

(51) Int. Cl. A61K 31/352; A61K 31/05

(52) U.S. Cl. CPC A61K 31/352; A61K 45/06; A61K 31/05

(57) ABSTRACT

Compositions comprising two or more cannabinoids, a terpene, and a flavonoid suitable for use in treating insomnia are disclosed. In preferred embodiments, the cannabinoids are cannabidiol and tetrahydrocannabinol, while the terpenes and flavonoids are selected from compounds that naturally occur in Cannabis extracts. Methods of treating insomnia by administering such compositions by a variety of routes are also disclosed.

### CROSS REFERENCE TO RELATED APPLICATIONS

This application claims priority to U.S. Provisional Application Ser. No. 62/562,817, filed Sep. 25, 2017; U.S. Provisional Application Ser. No. 62/562,823, filed Sep. 25, 2017; U.S. Provisional Application Ser. No. 62/562,826, filed Sep. 25, 2017; U.S. Provisional Application Ser. No. 62/562,832, filed Sep. 25, 2017; U.S. Provisional Application Ser. No. 62/562,836, filed Sep. 25, 2017; U.S. Provisional Application Ser. No. 62/562,840, filed Sep. 25, 2017; U.S. Provisional Application Ser. No. 62/562,845, filed Sep. 25, 2017; U.S. Provisional Application Ser. No. 62/562,850, filed Sep. 25, 2017; U.S. Provisional Application Ser. No. 62/562,853, filed Sep. 25, 2017, disclosures of each of which are hereby incorporated by reference in their entireties.

### FIELD OF THE DISCLOSURE

The present disclosure is directed to compositions capable of use to affect insomnia, along with methods of such use, delivery including devices and kits for delivery and making thereof.

### BACKGROUND OF THE DISCLOSURE

Insomnia and a host of related sleep disorders described herein afflict a significant, but largely unquantified, number of people at some point during their lives. Between 10% and 30% of adults have insomnia at any given point in time and up to half of people have insomnia at a point in each year. About 6% of people have insomnia that is not due to another problem and lasts for more than a month. People over the age of 65 are affected more often than younger people. Females are more often affected than males.

Insomnia, also known as sleeplessness, is a sleep disorder wherein people have trouble sleeping. They may have difficulty falling asleep or staying asleep if desired. Insomnia is typically followed by daytime sleepiness, low energy, irritability, and a depressed mood. It may result in an increased risk of motor vehicle collisions, as well as problems focusing and learning. Insomnia can be short term, lasting for days or weeks, or long term, lasting for more than a month.

Insomnia can occur independently or because of another problem. Conditions that can result in insomnia include, by way of example and not limitation, psychological stress, chronic pain, heart failure, hyperthyroidism, heartburn, restless leg syndrome, menopause, certain medications, and drugs such as caffeine, nicotine, and alcohol. Other risk factors include working night shifts and sleep apnea. Diagnosis is based on sleep habits and an examination to look for underlying causes. A sleep study may be done to look for underlying sleep disorders. Screening may be done with two questions: “do you have trouble sleeping?” and “do you have difficulty falling or staying asleep?”

Sleep hygiene and lifestyle changes are typically the first treatment for insomnia. Sleep hygiene includes a consistent bedtime, exposure to sunlight, a quiet and dark room, and regular exercise. Cognitive behavioral therapy may be added to this. While sleeping pills may help, they may be associated with injuries, dementia, and addiction, among other side effects. Medications are not recommended for more than four or five weeks. The effectiveness and safety of alternative medicine is to date unclear.

Further, as insomnia and sleep disorders vary significantly from individual to individual, traditional pharmaceutical efforts to treat these disorders have had varying degrees of success. Non-traditional treatments have also met with similarly varying results.

Accordingly, there is a need in the art for alternatives methods of treating insomnia and its related symptoms. It would further be advantageous if compositions and methods could be personalized to the affected individual to ensure success in treatment.

### BRIEF DESCRIPTION OF THE DISCLOSURE

The present disclosure provides a compositions and methods of using the compositions including a plurality of naturally occurring, synthetic or semisynthetic compounds to treat in a systematic way sleep disorders, and in particular, insomnia. Suitably, a variety of specific compositions are screened in a plurality of individuals and their sleep experience, and thus, their sleep disorders are evaluated against the compositions. Specific compositions are then selected or personalized to treat a given sleep disorder. In another suitable embodiment, biometric data from any given individual or group of individuals will be used to personalize the desired composition to treat that individual or group of individuals sleep disorder.

The present disclosure, therefore, is directed toward compositions, methods of making, delivering and therapeutic using the compositions for treatment of sleep disorders, and in particular, various forms of insomnia. The compositions comprise compounds found in cannabis and, optionally, synthetic or semisynthetic compounds as described herein.

It is further contemplated that a variety of compositions in a variety of dosage forms, both ethical pharmaceutical and nutraceutical, dietary supplements, functional foods, and the like are provided herein.

It is further contemplated that various kits, dosage devices and methods of personalizing the use of said devices for any given individuals or individual population will be provided for use with the compositions of the disclosure. One aspect of the present disclosure is directed to a composition. The composition comprises a cannabidiol (CBD), derivative, intermediate, or prodrug thereof, and combinations thereof in an amount from about 0.5 mg/mL to about 25 mg/mL, and combinations thereof; a tetrahydrocannabinol (THC), derivative, or intermediate thereof in an amount from about 0.5 mg/mL to about 30 mg/mL, and combinations thereof; at least one terpene; and at least one flavonoid.

Another aspect of the present disclosure is directed to a method of treating insomnia in a subject in need thereof. The method comprises administering to the subject a therapeutically effective amount of a composition. The composition comprises a cannabidiol (CBD), derivative, intermediate, or prodrug thereof, and combinations thereof in an amount of from about 0.5 mg/mL to about 25 mg/mL; a tetrahydrocannabinol (THC), derivative, or intermediate thereof, and combinations thereof in an amount of from about 0.5 mg/mL to about 30 mg/mL; at least one terpene; and at least one flavonoid.

In yet another aspect, the present disclosure is directed to a method of personalizing a composition for treating insomnia in a subject in need thereof. The method comprising: administering a first composition to the subject, the first composition comprising at least one compound obtained from a cannabis plant; performing or having performed a screening test to analyze one of more symptoms of insomnia; and adjusting one or more of: the type of the at least one compound in the first composition; the amount of the at least one compound in the first composition; and a ratio of the at least one compound and at least a second compound in the first composition to form a second composition. In some embodiments, the first composition comprises a cannabidiol (CBD), derivative, intermediate, or prodrug thereof, and combinations thereof in an amount of from about 0.5 mg/mL to about 25 mg/mL; a tetrahydrocannabinol (THC), derivative, or intermediate thereof, and combinations thereof in an amount of from about 0.5 mg/mL to about 30 mg/mL; at least one terpene; and at least one flavonoid.

In another aspect, the present disclosure is directed to a method of personalizing a composition for increasing total sleep time in a subject in need thereof. The method comprising: administering a first composition to the subject, the first composition comprising at least one compound obtained from a cannabis plant; performing or having performed a screening test to analyze one of more symptoms selected from the group consisting of sleep prolongation, minimized wakefulness, and delayed awaking; and adjusting one or more of: the type of the at least one compound in the first composition; the amount of the at least one compound in the first composition; and a ratio of the at least one compound and at least a second compound in the first composition to form a second composition.

Other aspects and features of the present disclosure will be in part apparent and in part pointed out hereinafter.

### DETAILED DESCRIPTION

Provided herein are compositions for treating insomnia and related sleep disorders and symptoms, methods of such use, delivery including devices and kits for delivery and making thereof.

### I. Compositions and Methods of Making

In an embodiment, the present disclosure provides a composition including at least one compound obtained from a cannabis plant. Cannabis is a genus of flowering plant in the family Cannabaceae, indigenous to Central Asia and the Indian subcontinent. While the number of species within the genus is disputed, three species have generally been recognized: Cannabis saliva, Cannabis indica and Cannabis ruderalis. C. ruderalis may be included within C. sativa, or all three may be treated as subspecies of the single species C. sativa.

C. sativa is an annual herbaceous plant in the Cannabis genus. It is a member of a small, but diverse, family of flowering plants of the Cannabaceae family. It has been cultivated and used as a source of industrial fiber, seed oil, food, recreation, in religious and spiritual ceremonies, and medicines. Each part of the plant is harvested differently, depending on the purpose of its use. For example, cannabis has long been used for hemp fiber, for hemp oils, for medicinal purposes, and as a recreational drug. Industrial hemp products are made from cannabis plants selected to produce an abundance of fiber. Further, some cannabis strains have been bred to produce minimal levels of tetrahydrocannabinol (THC), the principal psychoactive constituent. Many additional plants have been selectively bred to produce a maximum of THC (which is a cannabinoid), and other cannabinoids. THC can be obtained by curing the flowers, while hashish and hash oil can be extracted from the plant.

The present disclosure further provides methods allowing cannabis in the form a living plant to be converted into a composition of the present disclosure. The method of conversion typically involves one of: extraction, fraction or purification steps as described herein. More typically a combination of two or more such steps, more typically yet 2, 3, 4, 5, 6, 7, 8, 9 or even 10 individual steps described herein may be made. In suitable embodiments, a combination of separating the cannabis from the media in which it is grown, drying to reduce the water content, grinding to form a powder, extraction, and optionally, a fractionation or purification step is performed.

Suitably the cannabis is separated from the media in which it is grown and first dried and then ground. Once in the ground state, it is, optionally, sieved and finally the resins of the plant are extracted. These resins comprise the compositions of the present disclosure or additional synthetic or semisynthetic compounds may be added to the resins. Remembering that optional fractionation and purification steps are possible, the compositions of the disclosure may have compounds removed from the resin. At that point, again optionally, synthetic or semisynthetic compounds may be added to the resin to form the compositions of the present disclosure. Generally, the compositions include cannabinoids, terpenes and/or terpenoids, and flavonoids.

A cannabinoid is one of a class of diverse chemical compounds that acts on cannabinoid receptors such as CB1 and CB2 in cells that alter neurotransmitter release in the brain. Ligands for these receptor proteins include the endocannabinoids (produced naturally in the body by animals), the phytocannabinoids (found in cannabis and some other plants), and synthetic cannabinoids (manufactured artificially as set forth above). The most notable cannabinoid of the phytocannabinoids is tetrahydrocannabinol (THC), the primary psychoactive compound in cannabis. Cannabidiol (CBD) is another cannabinoid that is a major constituent of the plant. There are at least 113 different cannabinoids isolated from cannabis, exhibiting varied effects.

Synthetic cannabinoids and semisynthetic cannabinoids encompass a variety of distinct chemical classes: the classical cannabinoids structurally related to THC, the non-classical cannabinoids (cannabimimetics) including the aminoalkylindoles, 1,5-diarylpyrazoles, quinolines, and arylsulfonamides as well as eicosanoids related to endocannabinoids.

Tetrahydrocannabinol (THC) refers to a psychotropic cannabinoid and is the principal psychoactive constituent of cannabis. Its chemical name is (−)-trans-Δ9-tetrahydrocannabinol and the term “THC” is used to refer to isomers as well.

Like most pharmacologically-active secondary metabolites of plants, THC in cannabis is assumed to be involved in self-defense, perhaps against herbivores. THC also possesses high UV-B (280-315 nm) absorption properties, which, it has been speculated, could protect the plant from harmful UV radiation exposure.

Cannabidiol (CBD) is one of the active cannabinoids identified in cannabis. It is a major phytocannabinoid, by some accounts making up to 40% of the plant's extract. CBD does not appear to have any intoxicating effects such as those caused by THC in marijuana, but may have effects on anxiety, depression and have an anti-psychotic effect, and have effects on other comorbidities related to insomnia and sleep disorders such as pain and post-traumatic stress disorders commonly referred to as “PTSD”.

Cannabinol (CBN) is thought to be a non-psychoactive cannabinoid found only in trace amounts in cannabis and can be produced via oxidative degradation of THCA and THC. Pharmacologically relevant quantities are formed as a metabolite of tetrahydrocannabinol (THC). CBN acts as a partial agonist at the CB1 receptors, but has a higher affinity to CB2 receptors, however; with lower affinities in comparison to THC. Degraded or oxidized cannabis products, such as low-quality baled cannabis and traditionally produced hashish, are high in CBN, but modern production processes have been alleged to minimize the formation of CBN. Cannabinol has been shown to have analgesic properties. Unlike other cannabinoids, CBN does not stem from cannabigerol (CBG).

Cannabigerol (CBG) is thought to be a non-intoxicating cannabinoid found in the Cannabis genus of plants. CBG is the non-acidic form of cannabigerolic acid (CBGA), the parent molecule (“mother cannabinoid”) from which many other cannabinoids are obtained.

CBG has been found to act as a high affinity a2-adrenergic receptor agonist, moderate affinity 5-HT1A receptor antagonist, and low affinity CB1 receptor antagonist. It also binds to the CB2 receptor as an antagonist. CBG does not trigger THC-like activity in mice, rats, gerbils and non-human primates, consistent with it being non-intoxicating. Moreover, CBG was without effect up to 80 mg/kg in the mouse tetrad test of cannabimimetic activity (locomotor suppression, catalepsy, hypothermia and analgesia).

Cannabigerolic Acid (CBGA or CBG-A) is the alleged primordial phyto-cannabinoid. It is the alleged compound in cannabis from which all the plant's other naturally occurring cannabinoids are formed; without CBGA, the cannabis plant cannot produce its most useful compounds. It remains one of the most under-studied cannabinoids, with most of current research focusing on the purported healing properties of THC and CBD.

In suitable embodiments, the compositions generally include comprise a cannabidiol (CBD), derivative, intermediate, or prodrug thereof, and combinations thereof, and combinations thereof; a tetrahydrocannabinol (THC), derivative, or intermediate thereof, and combinations thereof; at least one terpene; and at least one flavonoid.

A. Cannabidiol (CBD), Derivative, Intermediate, or Prodrug Thereof

In an embodiment, the composition comprises a cannabidiol (CBD), derivative, intermediate, or prodrug thereof. In an embodiment, the cannabidiol (CBD) may be a plant-extract, a synthetic compound, or a semi-synthetic compound.

Suitable cannabidiol (CBD) derivatives, intermediates, or prodrugs include, without limitation, Cannabigerol-type (CBG): cannabigerol ((E)-CBG C-5), cannabigerol monomethyl ether ((E)-CBGM C-5 A), Cannabinerolsäure A ((Z)-CBGA C-5 A), Cannabigerovarin (((e)-CBGV C-3), Cannabigerolsäure A (e)-CBGA C-5 A), A Cannabigerolsäure monomethylether ((e)-CBGAM C-5 A), Cannabigerovarinsäure A ((e)-CBGVA-C 3 A); Cannabichromene-type (CBC): cannabichromene (CBC-C 5), Cannabichromensäure A (CBCA C-5 A), Cannabichromevarin (CBCVC-3), Cannabichromevarinsäure A (CBCVAC3 A); Cannabidiol-type (CBD), cannabidiol (CBD-C 5), cannabidiol monomethyl (CBDM-C 5), cannabidiol-C4 (CBD-C 4), Cannabidivarin (CBDV-C 3), Cannabidiorcol (CBD-C 1), cannabidiolic (CBDA C-5), Cannabidivarinsäure (CBDVA C-3); Cannabinodiollike (CBND): Cannabinodiol (CBND C-5), Cannabinodivarin (CBND C-3); Cannabinol-type (CBN): Cannabinol CBNC 5, cannabinol C4 (CBN-C4), Cannabivarin (CBN-C 3), cannabinol C2 (CBNC 2), Cannabiorcol (CBN-C 1), Cannabinolsäure A (C 5 CBNA-A), Cannabinolmethylether (CBNM C-5) Cannabitriol-type (CBT): (−)-(9R,10R)-trans-Cannabitriol ((−)-trans-CBTC 5), (+)-(9S,10S)-Cannabitriol ((+)-trans-CBT C-5), (±)-(9R,10S/9S,10R)-Cannabitriol ((±)-cis-CBT-C 5), (−)-(9R,10R)-trans [10-0-ethyl-cannabitriol] ((−)-trans- CBT-OEt-C 5), (±)-(9R,10R/9S,10S)-Cannabitriol-C3 ((±)-trans-CBT-C 3), 8,9-dihydroxy-Δ6a (10a) tetrahydrocannabinol (8,9-di-OH-CBT-C 5), cannabidiolic A (CBDA C-59-OH-CBT-05 ester), (−)-(6aR,9S,10S,10aR)-9,10-dihydroxyhexahydrocannabinol, Cannabiripsol Cannabiripsol-05, (−)-6a, 7,10a-trihydroxy-Δ9-tetrahydrocannabinol ((−)-Cannabitetrol), 10-oxo-Δ6a (10a) tetrahydrocannabinol (OTHC); Cannabielsoin-like (CBE): (5aS,6S,9R,9aR)-C 5-Cannabielsoin (CBEC-5), (5aS,6S,9R,9aR)-C 3-Cannabielsoin (CBE C-3), (5aS,6S,9R,9aR)-Cannabielsoinsäure A (CBEA-C 5 A), (5aS,6S,9R,9aR)-Cannabielsoinsäure B (CBEA-C 5 B), (5aS,6S,9R,9aR)-C3-Cannabielsoinsäure B (CBEA-C 3 B), Cannabiglendol-C3 (OH-iso-HHCV C-3), Dehydrocannabifuran (DCBF C-5), Cannabifuran (CBF-C 5); Isocannabinoide: (−)-Δ7-trans-(1R,3R, 6R)-Isotetrahydrocannabinol, (±)-Δ7-1,2-cis-(1R,3R,6S/1S,3S,6R)-Isotetrahydrocannabivarin, (−)-Δ7-trans-(1R,3R, 6R)-Isotetrahydrocannabivarin; Cannabicyclol-like (CBL): (±)-(1aS,3aR,8bR,8Cr-cannabicyclol (CBL-C 5), (±)-(1aS,3aR,8bR,8Cr-Cannabicyclolsäure A (CBLAC 5A) (±)-(1aS,3aR,8bR,8Cr-Cannabicyclovarin (CBLV C-3); Cannabicitran-type (CBT): Cannabicitran (CBT-C 5); Cannabichromanon-like (CBCN): Cannabichromanon (CBCN C-5), Cannabichromanon-C3 (CBCN C-3), Cannabicoumaronon (CBCON C-5), and combinations thereof. In a further embodiment, the cannabidiol (CBD), the derivative, the intermediate, or the prodrug thereof is selected from the group consisting of cannabidiolic acid (CBDA), cannabinol (CBN), cannabigerolic acid (CBGA), cannabinoid (CBG), cannabichromenic acid (CBCA), cannabichromene (CBC), cannabidivarin acid (CBDVA), cannabidivarin (CBDV), cannabigerovarin acid (CBGVA), and combinations thereof. In still a further embodiment, the cannabidiol (CBD), derivative, intermediate, or prodrug thereof is cannabidiol (CBD).

The composition may include a cannabidiol (CBD), derivative, intermediate, or prodrug thereof, and combinations thereof in an amount from about 0.5 mg/mL to about 25 mg/mL. In other embodiments, the composition comprises a cannabidiol (CBD), derivative, intermediate, or prodrug thereof, and combinations thereof in an amount of from about 1 mg/mL to about 25 mg/mL, from about 5 mg/mL to about 25 mg/mL, from about 5 mg/mL to about 20 mg/mL, from about 5 mg/mL to about 15 mg/mL, or from about from about 8 mg/mL to about 15 mg/mL. In some embodiments, the composition includes a cannabidiol (CBD), derivative, intermediate, or prodrug thereof, or combinations thereof in an amount of about 0.5 mg/mL, about 1 mg/mL, about 5 mg/mL, about 10 mg/mL, about 15 mL, about 20 mg/mL, or about 25 mg/mL.

B. Tetrahydrocannabinol (THC), Derivative, or Intermediate Thereof

In an embodiment, the compositions comprises a tetrahydrocannabinol (THC), derivative, or intermediate thereof. In an embodiment, the tetrahydrocannabinol (THC) may be a plant-extract, a synthetic compound, or a semi-synthetic compound.

Suitable tetrahydrocannabinol (THC) derivatives or prodrugs include, without limit, Tetrahydrocannabinol-like (THC): Δ9-tetrahydrocannabinol (Δ9-THC-C 5), Δ9-tetrahydrocannabinol-C4 (Δ9-THC-C 4), Δ9-tetrahydrocannabivarin (Δ9-THCV-C 3), Δ9-Tetrahydrocannabiorcol (Δ9-THCO C-1), Δ9-Tetrahydrocannabinolsäure (Δ9 THCA-C-5 A), Δ9-Tetrahydrocannabinolsäure B (Δ9 THCA-C-5 B), Δ9-Tetrahydrocannabinolsäure-C4 (Δ9 THCA-C-4 A and/or B), Δ9-Tetrahydrocannabivarinsäure A (Δ9-THCVA-C 3 A), Δ9-Tetrahydrocannabiorcolsäure (Δ9-THCOA-C 1 A and/or B), (−)-Δ8-trans-(6aR,10aR)-Δ8-tetrahydrocannabinol (Δ8-THC-C 5), (−)-Δ8-trans-(6aR, 10aR)-Tetrahydrocannabinolsäure A (Δ8-THCA-C 5 A); (−)-(6a S,10a R)-Δ9-tetrahydrocannabinol ((−)-cis-Δ9-THC-C 5). In a further embodiment, the tetrahydrocannabinol (THC), derivative, or intermediate thereof is selected from the group consisting of tetrahydrocannabinolic acid (THCA), tetrahydrocannabivarin carboxylic acid (THCVA), tetrahydrocannabivarin (THCV), and combinations thereof. In still a further embodiment, the tetrahydrocannabinol (THC), derivative, or intermediate thereof is tetrahydrocannabinol (THC).

The composition may include a tetrahydrocannabinol (THC), derivative, or intermediate thereof, and combinations thereof in an amount of from about 0.5 mg/mL to about 30 mg/mL. In other embodiments, the composition comprises the tetrahydrocannabinol (THC), derivative, or intermediate thereof, and combinations thereof in an amount of from about 1 mg/mL to about 30 mg/mL, from about 2 mg/mL to about 30 mg/mL, from about 5 mg/mL to about 30 mg/mL, from about 5 mg/mL to about 25 mg/mL, from about 5 mg/mL to about 20 mg/mL, from about 5 mg/mL to about 15 mg/mL, or about 5 mg/mL to about 10 mg/mL. In some embodiments, the composition comprises the tetrahydrocannabinol (THC), derivative, or intermediate thereof, and combinations thereof in an amount of about 0.5 mg/mL, about 0.75 mg/mL, about 1 mg/mL, about 5 mg/mL, about 10 mg/mL, about 15 mL, about 20 mg/mL, about 25 mg/mL, or about 30 mg/mL.

In an embodiment, the composition may include a ratio of the cannabidiol (CBD), derivative, intermediate, or prodrug thereof, and combinations thereof to the tetrahydrocannabinol (THC), derivative, or intermediate thereof, and combinations thereof may be from about 25:1 to about 1:25. In some embodiments, the ratio of the cannabidiol (CBD), derivative, intermediate, or prodrug thereof, and combinations thereof to the tetrahydrocannabinol (THC), derivative, or intermediate thereof, and combinations thereof may be about 25:1, about 20:1, about 15:1, about 10:1, about 5:1, about 1:1, about 1:5, about 1:10, about 1:15, about 1:20, or about 1:25. In a further embodiment, the ratio of the cannabidiol (CBD), derivative, intermediate, or prodrug thereof, and combinations thereof to the tetrahydrocannabinol (THC), derivative, or intermediate thereof, and combinations thereof may be about 1:1.

C. Terpene and Terpenoid

In an embodiment, the compositions includes at least one terpene and/or terpenoid. Terpenes and terpenoids are the primary constituents of the essential oils or many types of plants and flowers. Terpenes can be converted to a terpenoid, synthetic terpenoid or semisynthetic terpenoid by any known chemical reactions. In particularly suitable embodiments, the terpenes include, for example, alpha-Pinene, beta-Pinene, beta-Myrcene, alpha-Terpinene, Limonene, beta-Ocimene, Terpinolene, Linalool, Fenchyl Alcohol, Borneol Isomers, Alpha-Terpineol, Trans-caryophyllene, Alpha-humulene, Trans-nerolidol, Guaiol, Alpha-Bisabolol, and combinations thereof. Suitable terpenoids (and substances that include combinations of terpenes and terpenoids) include α-Pinene, β-Pinene, pine, linalool, llvender, black pepper, myrcene, musk, limonene, citrus, terpineol, lilac, nerolidol, wood bark, eucalyptol, mint, borneol, camphor; α-bisabolol, floral; D-3 Carene, pine, camphene, herbal, β-caryophyllene, Borneol, 1,8-cineole, camphene, humulene, limonene, linalool, nerolidol, pulegone, terpinolene, α-phellandrene, Δ3-carene, α-terpinene, β-phellandrene, cis-ocimene, terpinolene, β-caryophyllene, α-guaiene, humulene, δ-guaiene, elemene, guaiol, γ-eudesmol, β-eudesmol, agarospirol, bulnesol, and α-bisabolol.

The composition may include at least one terpene in an amount from about 0.001 mg/mL to about 1 mg/mL. In other embodiments, the composition may comprise at least one terpene in an amount of about 0.001 mg/mL to about 0.95 mg/mL, or about 0.001 mg/mL to about 0.9 mg/mL, or about 0.005 mg/mL to about 0.8 mg/mL. In some embodiments, the composition may comprise at least one terpene in an amount of about 0.01 mg/mL, about 0.15 mg/mL, about 0.02 mg/mL, about 0.25 mg/mL, about 0.03 mg/mL, about 0.35 mg/mL, about 0.04 mg/mL, about 0.45 mg/mL, about 0.05 mg/mL, about 0.55 mg/mL, about 0.06 mg/mL, about 0.65 mg/mL, about 0.07 mg/mL, about 0.75 mg/mL, about 0.08 mg/mL, about 0.085 mg/mL, about 0.09 mg/mL, about 0.95 mg/mL, or about 1 mg/mL. The concentrations listed is the total concentration of all the terpenes in the composition.

D. Flavonoid

In an embodiment, the composition includes at least one flavonoid. Flavonoids (or bioflavonoids) are a class of plant and fungus secondary metabolites. Main classes of flavonoids include: flavonoids or bioflavonoids, isoflavanoids (derived from 3-phenylchromen-4-one (3-phenyl-1,4-benzopyrone) structure) and neoflavonoids (derived from 4-phenylcoumarine (4-phenyl-1,2-benzopyrone) structure).

Suitable flavonoids include, without limit, 6-OH-Luteolin 4′-methyl ether-7-(2″-a-rhamnoside-6′″-acetyl-b-glucoside); 6-OHLuteolin 7-(6″-(E)-caffeoyl)-b-glucoside; Isoscutellarein 7-(2″-(6′″-acetyl)-b-allosyl)-bglucoside; Isoscutellarein 4′-methyl ether-7-(2″-(6″-acetyl)-b-allosyl)-b-glucoside; Apigenin 4′-(2″-(2′″-feruloyl-glucuronyl)-glucuronide); Apigenin 7-glucuronide-4′-(2″-(2′″-feruloyl-glucuronyl)-glucuronide); Apigenin 7-glucuronyl-4′-(2″-(2′″-E-pcoumaroyl-glucuronyl)-glucuronide); Luteolin 3′-b-glucoside-4′-(2″-a-rhamnosyl-bglucoside); Luteolin 3′,4′-di-b-glucoside; 5,7,4′-tri-OH-3′-OMe-Flavone 8-C-(2″-Ob-glucosyl-b-xyloside); 5,7-di-OH-3′-OMe-4′-Acetoxyflavone 8-C-(2″-O-b-glucosylb-xyloside); Iso-orientin 3′-methyl ether; 8-C-p-OH-Benzoyl-isovitexin 4′-glucoside; Apigenin 8-C-(2″-(4′″-acetyl-rhamnosyl)-glucoside); Spinosin; 6′″-Feruloyl-spinosin; Isoscoparin 7-glucoside; Carlinoside; Kaempferol 3-(6″-aarabinosyl-glucoside); Kaempferol 3-(6″ ‘ ’-a-arabinosyl-glucoside)-7-glucoside; Kaempferol 3-(2‘ ’-rhamnosyl-6‘ ’-malonyl-glucoside); Kaempferol 3-glucoside-7-(2″-(6′″-p-coumaroyl-glucosyl)-glucoside); 8-OMe-Kaempferol 3-(6″-malonyl-glucoside); Quercetin; Quercetin 4′-glucoside; Quercetin 3′-xyloside; Myricetin 3-(2″-acetyl-rhamnoside); Quercetin 3,4′-diglucoside; Isorhamnetin 3-rutinoside; Quercetin 3,7,4′-triglucoside; Isorhamnetin 3,7-diglucoside; Myricetin 3-(2″-rhamnosyl-glucoside); Myricetin 3′-(6″-p-coumaroyl-glucoside); Myricetin 7-(6″-galloyl-glucoside); Laricitrin 3-a-arabinofuranoside; Laricitrin 3-glucoside; Syringetin 3-(5″-glucosyl-a-arabinofuranoside); Syringetin 3-(6″-acetyl-glucoside); Syringetin 3-robinobioside; Syringetin 6-C-glucoside; 6,3′-di-OH-4,4′-di-OMe-5-Me-Aurone; 4,6,3′,4′-tetra-OMe-Aurone (Z-form); 4,6,3′,4′-tetra-OMe-Aurone (E-form); 6,3′,4′-tri-OH-4-OMe-5-Me-Aurone; Maesopsin; Maesopsin 6-O-glucoside (two diastereoisomers); Licoagroaurone; 3′-formyl-4′,6′-di-OH-2′-OMe-5-Me-Chalcone; Chalcononaringenin 2′,4′-diglucoside; 2′,4′-diOH-4′-OMe-6′-glucoside Dihydrochalcone; 2′-OH-3′,4′,6′-tri-OMe-Dihydrochalcone; Pelargonidin 3-glucoside-5-(6′″-acetyl-glucoside); Pelargonidin 3-(6″-feruloylglucoside)-5-(6′″-malonyl-glucoside); Cyanidin 3-(6″-malonyl-glucoside); Cyanidin 3-rutinoside; Cyanidin 3-(2″,3″-digalloyl-glucoside); Cyanidin 3,4′-diglucoside; Delphinidin 3-(6″-acetyl-galactoside); Delphinidin 3′-(2″-galloyl-6″-acetyl-galactoside); Peonidin 3-rutinoside; Petunidin 3,7-diglucoside; Petunidin 3-(6″-E-p-coumaroyl-glucoside)-5-(6′″-malonyl-glucoside); Malvidin 3-(6″-E-p-coumaroyl-glucoside)-5-glucoside; Malvidin 3-(6″-Z-p-coumaroyl-glucoside)-5-glucoside; Malvidin 3-rutinoside-5-glucoside; Malvidin 3-(6″-(4″″-malonylrhamnosyl)-glucoside)-5-(6′″-malonyl-glucoside); Apigeninidin 5-glucoside; Luteolinidin 5-glucoside; Carboxypyrano Pelargonidin 3-glucoside; Carboxypyrano Cyanidin 3-glucoside; Carboxypyrano Cyanidin 3-(6″-malonylglucoside;) Carboxypyrano Malvidin 3-glucoside; Judaicin 7-(6″-acetylglucoside); Tectorigenin 4′-(6″-glucosyl-glucoside); 7-OH-6′-OMe-3′,4′-methylenedioxyisoflavone 7-glucoside; Irisjaponin A; Irisjaponin B; Junipegenin B; Matteucinol 7-(6″-apiofuranosyl-b-glucoside); Hesperitin 7-(2″-galactosyl-6″-rhamnosyl-glucoside); Persicogenin 5,3′-di-OH-7,4′-di-OMeflavanone; Naringenin 7-glucoside; Naringenin 7-(6″-galloyl-glucoside); Taxifolin 4′-glucoside; Aromadendrin 7-glucoside; Ampelopsin 7-glucoside; 2″-Accallunin; 2R,3R-trans-aromadendrin 7-(6″-(4′″-OH-2′″-methylenebutanoyl)-glucoside); (2R,3S)-(b)-3′,5-di-OH-4′,7-di-OMe-Dihydroflavonol; 3-Desoxycallunin; Catechin 3-(6″-cinnamoyl-glucoside); Catechin 3-(2″-cinnamoyl-glucoside); Catechin 3-(2″,6″-dicinnamoyl-glucoside); Anadanthoside; Cajanin; Indicanine C; 6-(1,1-di-Me-allyl)-7,4′-di-OH-Flavan; 3-(4′-hydroxyphenyl)-5-methoxy-6-(3,3-dimethylallyl)-2″,2″-dimethylchromene-(5″,6″:8,7)-3-(propyl-2-one)-4H-1-benzo-2,3-Dihydropyran-2,4-dione; Maackianin 3-(6″-malonyl-glucoside); 3,4:8,9-Dimethylenedioxy-pterocarpan; Usararotenoid C; 12a-Epimillettosin; (p)-Usararotenoid-B; [Catechin 3-glucoside-(4a->8)-catechin 3-(2″-cinnamoyl-glucoside)]; [Catechin 3-glucoside-(4a->8)-epicatechin 3-(6″-cinnamoyl-glucoside)]; Amentoflavone; Aulacomnium-biaureusidin; Cupressuflavone 7,7″-dimethyl ether; 4,4′,6-tri-O-methyl-2-deoxymaesopsin-(2->7)-2,4,4′,6-tetra-O-Methylmaesopsin; Catechin-(4a->8)-pelargonidin 3-glucoside; 111. 2′,2″,2′″-tri-OH-4′,4′″-di-OMe-4-O-5′″-bichalcone (Rhuschalcone 1); Puerarin (Daidzein 8-C-glucoside); Calycosin; Isoneorautenol; Erybraedin A, and combinations thereof. In a further embodiment, the at least one flavonoid is selected from the group consisting of quercetin, apigenin-7-O-glucoside, luteolin, apigenin, kaempferol, cannflavin B, cannflavin A, myricetin, luteolin-7-O-glucoside, and combinations thereof.

The composition may include at least one flavonoid in an amount of from about 0.0001 mg/mL to about 0.01 mg/mL, including about 0.00025 mg/mL to about 0.0075 mg/mL, including about 0.0005 mg/mL to about 0.001 mg/mL, and including from about 0.0025 mg/mL to about 0.005 mg/mL. In some embodiments, the composition may comprise at least one flavonoid in an amount of about 0.0001 mg/mL, about 0.00025 mg/mL, about 0.0005 mg/mL, about 0.00075 mg/mL, about 0.001 mg/mL, about 0.0025 mg/mL, about 0.005 mg/mL, about 0.0075 mg/mL, or about 0.01 mg/mL. The concentrations listed is the total concentration of all the flavonoids in the composition.

In an exemplary embodiment, the composition of the present disclosure may include THC, CBDA, CBD, CBGA, CBG, CBCA, CBC, THCVA, THCV, CBDVA, CBDV, alpha-pinene, beta-pinene, beta-myrcene, limonene, linalool, fenchyl alcohol, borneol isomers, trans-caryophyllene, alpha-humulene, guaiol, alpha-bisabolol, cannflavin B, and cannflavin A.

In another exemplary embodiment, the composition of the present disclosure may include THC, CBDA, CBD, CBGA, CBG, CBCA, CBC, THCVA, THCV, CBDVA, CBDV, beta-myrcene, borneol isomers, trans-caryophyllene, alpha-humulene, guaiol, alpha-bisabolol, cannflavin B, and cannflavin A.

In an additional exemplary embodiment, the composition of the present disclosure may include THCA, THC, CBDA, CBD, CBN, CBGA, CBG, CBCA, CBC, THCVA, THCV, CBDVA, CBDV, fencyl alcohol, borneol isomers, trans-caryophyllene, alpha-humulene, guaiol, alpha-bisabolol, and cannflavin B.

In another exemplary embodiment, the composition of the present disclosure may include THCA, THC, CBDA, CBD, CBN, CBGA, CBG, CBCA, CBC, THCVA, THCV, CBDVA, CBDV, beta-myrcene, alpha-terpinene, terpinolene, borneol isomers, trans-caryophyllene, alpha-humulene, trans-nerolidol, and cannflavin B.

In an additional exemplary embodiment, the composition of the present disclosure may include THCA, THC, CBDA, CBD, CBN, CBGA, CBG, CBCA, CBC, THCVA, THCV, CBDVA, CBDV, beta-myrcene, linalool, fenchyl alcohol, borneol isomers, alpha-terpineol, trans-caryophyllene, alpha-humulene, trans-nerolidol, guaiol, alpha-bisabolol, cannflavin B, and cannflavin A.

In another exemplary embodiment, the composition of the present disclosure may include THCA, THC, CBDA, CBD, CBN, CBGA, CBG, CBCA, CBC, THCVA, THCV, CBDVA, CBDV, alpha-pinene, beta-pinene, beta-myrcene, limonene, beta-ocimene, terpinolene, linalool, alpha-terpineol, trans-caryophyllene, alpha-humulene, guaiol, alpha-bisabolol, cannflavin B, and cannflavin A.

In an additional exemplary embodiment, the composition of the present disclosure may include THCA, THC, CBD, CBN, CBGA, CBG, CBCA, CBC, THCVA, THCV, CBDVA, CBDV, beta-myrcene, limonene, borneol isomers, trans-caryophyllene, alpha-humulene, guaiol, alpha-bisabolol, cannflavin B, and cannflavin A.

In another exemplary embodiment, the composition of the present disclosure may include THCA, THC, CBD, CBN, CBGA, CBG, CBCA, CBC, THCVA, THCV, CBDVA, beta-myrcene, borneol isomers, trans-caryophyllene, alpha-humulene, trans-nerolidol, alpha-bisabolol, cannflavin B, and cannflavin A.

In an additional exemplary embodiment, the composition of the present disclosure may include THCA, THC, CBD, CBN, CBGA, CBG, CBCA, CBC, THCVA, THCV, CBDVA, CBDV, linalool, fenchyl alcohol, borneol isomers, alpha-terpineol, trans-caryophyllene, alpha-humulene, trans-nerolidol, guaiol, alpha-bisabolol, cannflavin B, and cannflavin A.

E. Matrix

The compositions described herein can, if desired, be formulated into a matrix. In certain embodiments, the matrix may be a lipid matrix. Suitable lipid matrices include, without limitation, natural and/or synthetic oils, fatty acids and their derivatives, glycerides, fatty acid esters, glycolized fatty acid esters, fatty alcohols, sterols, waxes, hard fat, and/or combination thereof.

Suitable natural oils include, without limitation, vegetable oil such as sunflower oil, olive oil, groundnut oil, and palm oil, as well as hydrogenated vegetable oils, including hydrogenated cottonseed oil. Suitable synthetic oils include, without limit, hydrophobic silicone, cyclomethicones, petroleum waxes or jellies, linear alkanes, lipophilic organic fluorinated oils, perhydrosqualene and/or mixtures thereof.

Suitable fatty acids include, without limitation, stearic acid, benzoic acid, citric acid, iumaric acid, lactic acid, and maleic acid. Exemplary glycerides include, without limitation, monoglycerides, diglycerides, triglycerides, and combinations thereof, etc. with saturated or unsaturated chains having carbon numbers from C₆ to C₄₀, e.g. C₁₈ to C₂₄, C₈ to C₃₂, C₁₀ to C₂₄, C₁₀ to C₁₈, C₁₂ to C₁₈, etc.), hemisynthetic glycerides or glyceride derivatives with saturated or unsaturated medium to long chain lengths. Exemplary long-chain fatty acid esters include, without limitation, glyceryl monooleate, glyceryl monostearate, glycerol behenate, glycerol monostearate, glyceryl palmitostearate, mixtures of mono-, di-, and trialkyl glycerides, including mixtures of glyceryl mono-, di-, and tribehenate (e.g. available commercially as COMPRITOL products from Gattetosse), glyceryl tristearate, and glyceryl tripalmitate. Exemplary non-neutralized fatty acid include, without limitation, fatty acids with linear chains with carbon numbers ranging from C₄ to C₁₈, for example such as myristic acid, lauric acid, palmitic acid, or oleic acid and mixtures thereof.

Suitable short to medium chain fatty acid esters include, without limitation, isopropyl palimitate, isopropyl myristate, triethyl citrate, lecithin, triacetin, and dibutyl sebacate. Esters of fatty acids with carbon numbers from C₆ to C₁₂ with glycols, e.g. ethylene glycol, propylene glycol, may also be used. Glycolized fatty acid esters include, without limitation, polyethylene glycol stearate and polyethylene glycol distearate.

Suitable sterols include, without limitation, cholesterol and its esters. Suitable waxes include, without limitation, Carnauba wax, Candelilla wax, Alfa wax, vegetable waxes, rice wax, hydrogenated jojoba wax or floral absolute waxes, beeswaxes and modified beeswaxes, microcrystalline wax, and paraffin wax. Suitable fatty alcohols include fatty alcohols with high molecular weight (e.g. cetanol, myristoyl alcohol, stearyl alcohol).

Esters of acids and alcohols with high molecular weight include, without limitation, esters of linear and saturated acids with even carbon numbers from C₁₄ to C₂₀, and linear and saturated alcohols with even carbon numbers from C₁₄ to C₃₂.

The matrix material may include mixtures of materials, such as mixtures of any of the foregoing.

In certain embodiments, lipid matrix materials include an alkyl-containing glycerol such as a mixture of mono-, di- and tri glyceryl behenates (commercially available as COMPRITOL 888, SUPPOCIRE, a semi-synthetic glyceride base comprising saturated C₁₀-C₁₈ triglyceride fatty acids and PRECIROL (glyceryl distearate) from Gattefose Corporation of Westwood, N.J.); and hydrogenated cottonseed oil (commercially available as LUBR1TAB from Edward Mendell Co. of Patterson, N.Y.).

The lipid matrix may also include clays or their oily dispersions, gums of phenylated silicones, starches, and/or fat structuring agents for the purpose of adjusting consistency. The lipid matrix may also include a certain number of compounds such as mineral fillers, to modulate density and plasticity. The mineral fillers may be, for example, talc and/or kaolin.

The amount of lipid matrix present in the solid lipid particles may be at a weight percent of the total weight of the solid lipid particles of from about 1% to about 90%, from about 1% to about 75%, about 25% to about 70%, or any value or range in between. In some embodiments, the amount of lipid matrix present may be about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90%.

F. Pharmaceutical Excipient

The compositions described herein can, if desired, include one or more pharmaceutically acceptable excipients. Suitable excipients include, without limitation, binders, disintegrants, taste enhancers, solvents, thickening agents, penetration enhancers, wetting agents, lubricants, emollients, substances added to mask or counteract a disagreeable odor, fragrances or taste, and substances added to improve appearance or texture of the composition. Any such excipients can be used in any dosage forms according to the present disclosure. The foregoing classes of excipients are not meant to be exhaustive but merely illustrative as a person of ordinary skill in the art would recognize that additional types of excipients could be used to achieve the desired goals for delivery of the disclosed compositions.

Compositions of the disclosure containing excipients can be prepared by any technique known to a person of ordinary skill in the art of pharmacy, pharmaceutics, drug delivery, pharmacokinetics, medicine or other related discipline that comprises admixing an excipient with a drug or therapeutic agent.

In an embodiment, the disclosed compositions can be combined with a penetration enhancing agent for transdermal or topical delivery. Suitable penetration enhancing agents include, without limitation, C₈-C₂₂ fatty acids such as isostearic acid, octanoic acid, and oleic acid; C₈-C₂₂ fatty alcohols such as oleyl alcohol and lauryl alcohol; lower alkyl esters of C₈-C₂₂ fatty acids such as ethyl oleate, isopropyl myristate, butyl stearate, and methyl laurate; di(lower)alkyl esters of C₆-C₂₂ diacids such as diisopropyl adipate; monoglycerides of C₈-C₂₂ fatty acids such as glyceryl monolaurate; tetrahydrofurfuryl alcohol polyethylene glycol ether; polyethylene glycol, propylene glycol; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; dimethyl sulfoxide; glycerol; ethyl acetate; acetoacetic ester; N-alkylpyrrolidone; and terpenes.

In another embodiment, the disclosed compositions can be combined with a thickening or gelling agent. Suitable thickening agents (aka gelling agents) which may be used herein include, without limitation, anionic polymers such as polyacrylic acid (CARBOPOL by Noveon, Inc., Cleveland, Ohio), carboxypolymethylene, carboxymethylcellulose and the like, including derivatives of CARBOPOL polymers, such as CARBOPOL Ultrez 10, CARBOPOL 940, CARBOPOL 941, CARBOPOL 954, CARBOPOL 980, CARBOPOL 981, CARBOPOL ETD 2001, CARBOPOL EZ-2 and CARBOPOL EZ-3, and other polymers such as PEMULEN polymeric emulsifiers, and NOVEON polycarbophils. Additional thickening agents, enhancers and adjuvants may generally be found in Remington's The Science and Practice of Pharmacy as well as the Handbook of Pharmaceutical Excipients, Arthur H. Kibbe ed. 2000 (the disclosures of which are hereby incorporated by reference in their entirety). Thickening agents or gelling agents are present in an amount sufficient to provide the desired rheological properties of the composition. Illustratively, one or more pharmaceutically acceptable thickening agent or gelling agent are present in a total amount by weight of about 0.1%, about 0.25%, about 0.5%, about 0.75%, about 1%, about 1.25%, about 1.5%, about 1.75%, about 2.0%, about 2.25%, about 2.5%, about 2.75%, about 3.0%, about 3.25%, about 3.5%, about 3.75%, about 4.0%, about 4.25%, about 4.5%, about 4.75%, about 5.0%, about 5.25%, about 5.5%, about 5.75%, about 6.0%, about 6.25%, about 6.5%, about 6.75%, about 7.0%, about 7.25%, about 7.5%, about 7.75%, about 8.0%, about 8.25%, about 8.5%, about 8.75%, about 9.0%, about 9.25%, about 9.5%, about 9.75%, about 10%, about 10.25%, about 10.5%, about 10.75%, about 11%, about 11.25%, about 11.5%, about 11.75%, about 12%, about 12.25%, about 12.5%, about 12.75%, about 13%, about 13.25%, about 13.5%, about 13.75%, about 14%, about 14.25%, about 14.5%, about 14.75%, or about 15%. In some embodiments, the composition may comprise one or more pharmaceutically acceptable thickening agent or gelling agent in an amount from about 0.1% to about 15%, from about 1% to about 15%, or from about 1% to about 10%.

Compositions described herein may optionally comprise one or more pharmaceutically acceptable wetting agents as excipients. Suitable surfactants include, without limitation, quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., LABRASOL of Gattefosse), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., TWEEN80 of ICI), propylene glycol fatty acid esters, for example propylene glycol laurate (e.g., LAUROGLYCOLof Gattefosse), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total from about 0.25% to about 15%, from about 0.4% to about 10%, or from about 0.5% to about 5%, of the total weight of the composition. In some embodiments, one or more pharmaceutically acceptable wetting agents are present in a total amount by weight of about 0.25%, about 0.5%, about 0.75%, about 1%, about 1.25%, about 1.5%, about 1.75%, about 2.0%, about 2.25%, about 2.5%, about 2.75%, about 3.0%, about 3.25%, about 3.5%, about 3.75%, about 4.0%, about 4.25%, about 4.5%, about 4.75%, about 5.0%, about 5.25%, about 5.5%, about 5.75%, about 6.0%, about 6.25%, about 6.5%, about 6.75%, about 7.0%, about 7.25%, about 7.5%, about 7.75%, about 8.0%, about 8.25%, about 8.5%, about 8.75%, about 9.0%, about 9.25%, about 9.5%, about 9.75%, about 10%, about 10.25%, about 10.5%, about 10.75%, about 11%, about 11.25%, about 11.5%, about 11.75%, about 12%, about 12.25%, about 12.5%, about 12.75%, about 13%, about 13.25%, about 13.5%, about 13.75%, about 14%, about 14.25%, about 14.5%, about 14.75%, or about 15%.

Compositions described herein may optionally comprise one or more pharmaceutically acceptable lubricants (including anti-adherents and/or glidants) as excipients. Suitable lubricants include, without limitation, either individually or in combination, glyceryl behanate (e.g., COMPRITOL888); stearic acid and salts thereof, including magnesium (magnesium stearate), calcium and sodium stearates; hydrogenated vegetable oils (e.g., STEROTEX; colloidal silica; talc; waxes; boric acid; sodium benzoate; sodium acetate; sodium fumarate; sodium chloride; DL-leucine; PEG (e.g., CARBOWAX4000 and CARBOWAX6000); sodium oleate; sodium lauryl sulfate; and magnesium lauryl sulfate. Such lubricants, if present, constitute in total from about 0.1% to about 10%, from about 0.2% to about 8%, or from about 0.25% to about 5%, of the total weight of the composition. In some embodiments, one or more pharmaceutically acceptable lubricants are present in a total amount by weight of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, or about 10.0%.

In another embodiment, the compositions described herein may optionally comprise an emollient. Suitable emollients include, without limitation, mineral oil, mixtures of mineral oil and lanolin alcohols, cetyl alcohol, cetostearyl alcohol, petrolatum, petrolatum and lanolin alcohols, cetyl esters wax, cholesterol, glycerin, glyceryl monostearate, isopropyl myristate, isopropyl palmitate, lecithin, allyl caproate, althea officinalis extract, arachidyl alcohol, argobase EUC, butylene glycol dicaprylate/dicaprate, acacia, allantoin, carrageenan, cetyl dimethicone, cyclomethicone, diethyl succinate, dihydroabietyl behenate, dioctyl adipate, ethyl laurate, ethyl palmitate, ethyl stearate, isoamyl laurate, octanoate, PEG-75 lanolin, sorbitan laurate, walnut oil, wheat germ oil, super refined almond, super refined sesame, super refined soyabean, octyl palmitate, caprylic/capric triglyceride and glyceryl cocoate. An emollient, if present, is present in the compositions described herein in an amount of from about 1% to about 30%, from about 3% to about 25%, or from about 5% to about 15%, by weight. In some embodiments, the one or more emollients are present in a total amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%, by weight.

In one embodiment, the compositions described herein comprise an antimicrobial preservative. Suitable antimicrobial preservatives include, without limitation, acids, benzoic acid, phenolic acid, sorbic acids, alcohols, benzethonium chloride, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, propylparaben, sodium propionate, or thimerosal. The antimicrobial preservative, if present, is present in an amount of from about 0.1% to about 5%, from about 0.2% to about 3%, or from about 0.3% to about 2%, by weight. In some embodiments, the anti-microbial preservative, if present, is present in an amount of about 0.1%, about 0.2%, about 0.4%, about 0.6%, about 0.8%, about 1%, about 1.2%, about 1.4%, about 1.6%, about 1.8%, about 2%, about 2.2%, about 2.4%, about 2.6%, about 2.8%, about 3.0%, about 3.2%, about 3.4%, about 3.6%, about 3.8%, about 4%, about 4.2%, about 4.4%, about 4.6%, about 4.8%, or about 5%.

Compositions described herein may optionally include one or more emulsifying agents. Suitable emulsifying agents can come from any class of pharmaceutically acceptable emulsifying agents including carbohydrates, proteins, high molecular weight alcohols, wetting agents, waxes and finely divided solids. The optional emulsifying agent, if present, is present in a composition in a total amount of from about 1% to about 15%, from about 1% to about 12%, from about 1% to about 10%, or from about 1% to about 5% by weight of the composition. In some embodiments, one or more emulsifying agents are present in a total amount by weight of about 1%, about 2%, about 3%, about 4%, about 5%, about 6%, about 7%, about 8%, about 9%, about 10%, about 11%, about 12%, about 13%, about 14%, or about 15%.

In another embodiment, the water immiscible solvent includes propylene glycol, and is present in a composition in an amount of from about 1% to about 99%, by weight of the composition. In some embodiments, the water immiscible solvent includes propylene glycol, and is present in a composition in an amount of about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%. In other embodiments, the composition includes propylene glycol in an amount of about 1% to about 99%, from about 1% to about 90%, from about 1% to about 80%, from about 1% to about 70%, from about 1% to about 60%, from about 1% to about 50%, from about 1% to about 40%, from about 1% to about 30%, from about 1% to about 20%, or from about 1% to about 10%.

Compositions described herein may optionally include one or more binding agents. Binding agents may be either dry or wet. Dry binding agents may include, without limit, simple and complex carbohydrates (e.g., sucrose, glucose, fructose, maltose, lactose, maltodextrins, starch, modified starches, mannitol, sorbitol, maltitol, xylitol, and erythritol), cellulose, and cellulosic derivatives (e.g., microcrystalline cellulose, carboxymethyl cellulose, and hydroxyethyl cellulose). Wet binder agents may include, without limit, polyvinyl pyrrolidone, methycellulose, hydroxypropyl cellulose, hydroxypropyl methylcellulose, carboxymethylcellulose, xanthan gum, carrageenan gum, locust bean gum, alginates, and acacia. Depending on the desired result, a person of ordinary skill in the art of pharmacy, pharmaceutics, drug delivery, pharmacokinetics, medicine, or other related discipline that comprises admixing an excipient with a drug or therapeutic agent to a composition would be able to select the appropriate binding agent and the relative concentration of the binding agent.

In another embodiment, the compositions described herein may contain disintegrants, such as sodium starch glycolate, crosspovidone, crosscarmellose, microcrystalline cellulose, and starch. Depending on the desired result, a person of ordinary skill in the art of pharmacy, pharmaceutics, drug delivery, pharmacokinetics, medicine, or other related discipline that comprises admixing an excipient with a drug or therapeutic agent to a composition would be able to select the appropriate disintegrant and the relative concentration of the disintegrant.

In a further embodiment, the compositions disclosed herein may contain lubricants, such as magnesium stearate, stearic acid and its pharmaceutically acceptable salts, talc, vegetable oils, and waxes. Depending on the desired result, a person of ordinary skill in the art of pharmacy, pharmaceutics, drug delivery, pharmacokinetics, medicine, or other related discipline that comprises admixing an excipient with a drug or therapeutic agent to a composition would be able to select the appropriate lubricant and the relative concentration of the lubricant.

Compositions described herein may also optionally include one or more taste enhancers, such as sweeteners, including aspartame, acesulfame potassium, sucralose and saccharin or taste masking agents, such as flavorings. Depending on the desired result, a person of ordinary skill in the art of pharmacy, pharmaceutics, drug delivery, pharmacokinetics, medicine or other related discipline that comprises admixing an excipient with a drug or therapeutic agent to a composition would be able to select the appropriate taste enhancer or taste making agent and the relative concentration of the taste enhancer or taste masking agent.

Compositions described herein may also optionally include one or more vitamins or nutritional additives. Suitably nutritional additives comprise, without limit, essential nutrients including vitamins, dietary minerals amino acids and fatty acids vitamin A, vitamin B1, vitamin B2, vitamin B3, vitamin B5, vitamin B6, vitamin B7, vitamin B9, vitamin B 12, vitamin C, vitamin D, vitamin E, vitamin K calcium, phosphorus, potassium, sulfur, sodium, chlorine, magnesium, iron, cobalt, copper, zinc, molybdenum, iodine, selenium, manganese, nickel, chromium, fluorine, boron, strontium, histidine, isoleucine, leucine, lysine, methionine, cysteine, phenylalanine, tyrosine, threonine, tryptophan, valine, alpha-linoleic acid, and linoleic acid.

In an additional embodiment, the compositions described herein may be formulated as a nutraceutical, a dietary supplement, or a functional food.

### III. Methods of Treatment

In an aspect, the present disclosure provides a method of treating insomnia in a patient in need thereof. The method generally includes administering to the subject a therapeutically effective amount of a composition as described herein. In suitable embodiments, the compositions generally include a cannabidiol (CBD), derivative, intermediate, or prodrug thereof, and combinations; a tetrahydrocannabinol (THC), derivative, or intermediate thereof, and combinations thereof; at least one terpene; and at least one flavonoid.

Insomnia, as used herein, may refer to acute insomnia, chronic insomnia, comorbid insomnia, onset insomnia, maintenance insomnia, adjustment insomnia, idiopathic insomnia, non-organic specific insomnia, organic specific insomnia, paradoxical insomnia, psychophysiological insomnia, sleep hygiene insomnia, adjustment insomnia, behavioral insomnia of childhood, idiopathic insomnia, insomnia due to medical condition(s), and insomnia due to mental disorder(s), or a sleep related disorder including, without limit, REM sleep behavior disorder, sleep talking, sleep walking, nightmares, shift work disorder, delayed sleep phase disorder, excessive daytime sleepiness disorder, excessive sleepiness, narcolepsy, excessive sleepiness, restless leg syndrome, periodic limb movements, teeth grinding, and mental health daily.

In an aspect, the compositions of the present disclosure may improve total time asleep, rested after sleep, sleep quality, and reduction of latency of sleep.

Suitable dosages may be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

Suitable amounts of the cannabidiol (CBD), derivative, intermediate, or prodrug thereof, and combinations thereof and the tetrahydrocannabinol (THC), derivative, or intermediate thereof, and combinations thereof use in the dosage forms of the present disclosure will depend upon many factors including, for example, age and weight of an individual, specific cannabidiol (CBD), derivative, intermediate, or prodrug thereof, and combinations thereof and the tetrahydrocannabinol (THC), derivative, or intermediate thereof, and combinations thereof to be used, nature of a composition, whether the composition is intended for direct administration or is a concentrate, and combinations thereof. Ultimately, a suitable amount can be readily determined by one skilled in the art. For example, one skilled in the art can begin with a low amount that can be increased until reaching the desired result or effect. Alternatively, one skilled in the art can begin with a high dosage that can be decreased until reaching a minimum dosage needed to achieve the desired result or effect.

Suitable subjects include, but are not limited to, a human, a livestock animal, a companion animal, a lab animal, and a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In another embodiment, the animal is a laboratory animal. Non-limiting examples of a laboratory animal may include rodents, canines, felines, and non-human primates. In certain embodiments, the animal is a rodent. In a further embodiment, the subject is human.

As used herein, an “individual in need” refers to an individual at risk for or having a medical need such as those described herein. Additionally, an “individual in need” is also used herein to refer to an individual at risk for or diagnosed by a medical professional as having a condition described herein. It should be understood that the terms “individual” and “subject” are used interchangeably throughout the description.

In certain aspects, the composition disclosed herein may be administered to a subject. Administration is performed using standard techniques, including transdermal, parenteral, topical, oral, buccal, sublingual, injection, rectal, vaginal, ocular, nasal, or inhalation.

In one embodiment, compositions described herein are suitable for transdermal administration. In another embodiment, transdermally administrable compositions are adapted for administration in and/or around the abdomen, back, chest, legs, arms, scalp or other suitable skin surface and may include formulations of the compositions disclosed herein is administered in patches, ointments, creams, suspensions, lotions, pastes, gels, sprays, foams, or oils.

In another embodiment, compositions described herein which are transdermally administrable include formulations of the compositions disclosed herein is placed in a glycol or gel formulation.

In one embodiment, compositions described herein are suitable for parenteral administration. In another embodiment, parenteral administrable compositions are adapted for administration by a needle, syringe, or insertion of an indwelling catheter. In some embodiments, the compositions of the present disclosure may, for example, be injected or infused into the epidural space, intracerebral, or intracerebroventricular.

In one embodiment, compositions described herein are suitable for topical administration. In another embodiment, topical administrable compositions are adapted for administration in and/or around the abdomen, back, chest, legs, arms, scalp, or other suitable skin surface and may include formulations of the compositions disclosed herein is administered in patches, ointments, creams, suspensions, lotions, pastes, gels, sprays, foams, or oils.

In another embodiment, the compositions described herein are suitable for oral administration. In another embodiment, compositions described herein that are orally administrable include formulations of the compositions disclosed herein is administered in tablets, capsules, gel capsules, suspensions, emulsions, syrups or liquids. In an additional embodiment, the composition maybe formulated as extended release, controlled release, or long acting tablet, or capsule. In a further embodiment, the oral dosage form may be enteric coated using compositions and techniques known to a person of ordinary skill in the art.

In one embodiment, compositions described herein are suitable for buccal administration. In another embodiment, compositions described herein that are bucally administrable may include formulations of the compositions disclosed herein is administered in lozenges, troche, sprays, gels, pastes, dissolvable tablets, dissolvable strips, or snuff pack.

In one embodiment, compositions described herein are suitable for sublingual administration. In another embodiment, compositions described herein that are sublingually administrable may include formulations of the compositions disclosed herein is administered in lozenges, sprays, gels, pastes, dissolvable tablets, dissolvable strips, or snuff pack.

In one embodiment, compositions described herein are suitable for injectable administration. In another embodiment, compositions described herein that are administrated by injection may include formulations of the compositions disclosed herein is administered as an intravenous, intrathecal, subcutaneous, or depot injection.

In one embodiment, compositions described herein are suitable for rectal administration. In another embodiment, compositions described herein that are rectally administrable may include formulations of the compositions disclosed herein is placed in suppositories, ointments, creams, suspensions, solutions, lotions, pastes, gels, sprays, foams, or oils.

In one embodiment, compositions described herein are suitable for vaginal administration. In another embodiment, compositions described herein that are vaginally administrable may include formulations of the compositions disclosed herein is placed in suppositories, ointments, creams, suspensions, solutions, lotions, pastes, gels, sprays, foams, or oils.

In one embodiment, compositions described herein are suitable for ocular administration. In another embodiment, compositions described herein that are ocularly administrable may include formulations of the compositions disclosed herein is placed in ointments, suspensions, solutions, gels, or sprays.

In one embodiment, compositions described herein are suitable for nasal administration. In another embodiment, compositions described herein that are nasally administrable may include formulations of the compositions disclosed herein is placed in ointments, suspensions, solutions, lotions, pastes, gels, sprays, or mists.

In one embodiment, compositions described herein are suitable for inhalation administration. In another embodiment, compositions described herein that are inhaled administrable may include formations of the compositions disclosed herein is placed in an inhaler, vaporizer, vape pen, or the like.

In one embodiment, compositions described herein are suitable for gastric tube administration. The compositions of the present disclosure may be administered directly into the stomach by gastric tube feeding or gastrostomy. The compositions of the present disclosure may be placed into the small intestines, as with a duodenal feeding tube and enteral nutrition.

II. Delivery Devices or Kits

The compositions can be administered using any device or kit for delivery known in the art.

In an embodiment, the compositions described herein may be provided in a device for delivery to a subject in need thereof. The device may include any container suitable for holding a maximum amount of the composition, a provisioning mechanism for providing a dose of the composition to the subject, and a metering system for transporting the composition to the provisioning mechanism, such that the amount of composition delivered to the subject is controlled by the metering system. The device may deliver any amount of the composition held in the container. In a further embodiment, the amount delivered to the subject is less than the maximum amount held in the container. In some embodiments, the amount delivered to the subject is the same as the maximum amount held in the container.

The delivery of the composition from the device to the subject may be controlled by the subject. In an alternative embodiment, the delivery of the composition from the device to the subject is not controlled by the subject.

In another embodiment, the device may include a container suitable for holding a maximum amount of the composition; a mechanism for opening the container and allowing delivery of the composition to the consumer; and a label; such that the amount of composition held by the container is described by the label.

In an embodiment, the device(s) disclosed herein are programmed to limit the amount of the composition that can be delivered to a subject in need thereof within a specified time frame. Thus, the subject cannot consume more of the composition described herein during the specified time frame.

IV. Personalized methods

In yet other embodiments, the present disclosure is directed to methods for personalizing compositions for treating insomnia and its symptoms in a subject in need thereof. As used herein, a “subject in need” refers to an individual at risk for or having insomnia or related sleep disorder and/or symptoms (e.g., sleep prolongation, sleep quality, minimized wakefulness, and/or delayed awaking). As such, in some embodiments, the methods disclosed herein are directed to a subset of the general population such that, in these embodiments, not all of the general population may benefit from the methods. Based on the foregoing, because some of the method embodiments of the present disclosure are directed to specific subsets or subclasses of identified individuals (that is, the subset or subclass of subjects “in need” of assistance in addressing one or more specific conditions noted herein), not all individuals will fall within the subset or subclass of individuals as described herein. In particular, the subject in need is a human. The subject in need can also be, for example, a research animal such as, for example, a non-human primate, a mouse, a rat, a rabbit, a cow, a pig, and other types of research animals known to those skilled in the art.

Generally, the methods include administering a composition as described herein to the subject; performing or having performed a screening test to analyze one or more symptoms of insomnia; and adjusting one or more of the type of compound in the composition, an amount of at least one compound in the composition, and a ratio of at least two compounds of the composition to form a new second composition.

The screening test for analyzing one or more symptoms of insomnia can be any screening test or method known in the art capable of measuring changes in identified outcomes as a result of composition of the present disclosure versus placebo and/or composition of the present disclosure versus convention sleep drug. By way of example, without limitation, the screening test can include one or more of a self-reporting study, diary, survey, or biometric test. In particular, the screening test or method includes analyzing one or more of the following: measuring sleep/functional outcomes; diary/actigraph: changes in sleep latency, number of times awaken, time awoken, total sleep time, changes in QoL; polysomnography; EEG: changes in sleep stages: i.e., changes in latency to REM sleep, time in REM sleep, time in SWS, time in Stage 1-2 sleep etc.; EOG: changes in sleep stages: Latency and time (REM vs. NREM); EMG: changes periodic limb movements, restless leg syndrome, etc.; ECG: changes in heart rhythm; pulse oximetry: changes in respiratory airflow; imaging; changes in regional cerebral blood flow (SPECT) at night or during the day; ligand neuroimaging studies (PET or SPECT ligand); Multiple Sleep Latency Test (MSLT) (changes in level of daytime sleepiness); Maintenance of Wakefulness Test (MWT) (changes and levels of alertness); Driving Simulator-measure of changes in alertness; Questionnaire/other written assessments; changes in: Epworth Sleepiness Scale; Berlin Questionnaire® (Sleep apnea); Stanford Sleepiness Scale; Sam-Perelli fatigue rating; Insomnia Survey Index (ISI); Morning Eveningness Questionnaire (MEQ); Pittsburgh Sleep Quality Index (PSQI); Toronto Hospital Alertness Test (THAT); Athens Insomnia Scale; Center for Epidemiologic Studies in Depression Scale; Fatigue Severity Scale; Changes in diagnostic qualifications of insomnia disorders and related according to the DSM 5; or measuring secondary outcome changes—some possibly related to sleep: blood sugar, weight, cortisol, working memory, emotion discrimination and expression, reward processing; changes in prescription medication use (i.e. changes in use of ‘Z’ drugs); Dim Light Melatonin Onset (DLMO) Test-measures melatonin in saliva within a specified time (i.e., 8 pm to 3 am); Up- and/or down-regulation of specific receptors (CB1, CB2, GABAa, 5-HT1a, adenosine A2A receptors, etc.) or neurotransmitters/enzymes (fatty acid amide hydrolase (FAAH), anandamide, 2-AG, Ach, AchE, 5-HT, GABA, etc.) in test subject or cell culture (including IPSCs); Ketamine or pentobarbital-induced sleep tests with study drug and measure outcomes including: Sleep latency, EEG, EMG, locomotor activity, body temperature, motor coordination, EEG; locomotor activity (possibly including motor coordination); Memory tests (i.e., Morris water maze (MWM), novel object recognition); Emotion/fear regulation (i.e. fear conditioning test); Anxiety/depression tests (i.e. EPM, or tail hang test); EMG; and measure action potentials in brain slices. Further exemplary testing or methods include radiological methods such as magnetic resonance imagery on the subject.

Biometric testing typically includes gathering a biological sample from the sample prior to, during, or following administration of the composition. Biological samples as known in the art can be used, including, without limitation, blood, urine, plasma, cerebral spinal fluid, saliva, and combinations thereof.

The typical symptoms of insomnia being screened for include any of the symptoms identified above with respect to the various types of insomnia and related sleep disorders. In particular, symptoms for screening include sleep prolongation, sleep quality, minimized wakefulness, and/or delayed awaking.

To determine what and how to adjust types, amounts and ratios of compounds in the composition, the methods may further include obtaining analytical data to determine the presence, absence or amount of compounds described herein at one or more points in time characterized as prior to, during or following administration of the composition to the subject.

While adjusting the type of compound can include exchanging one compound in a class (e.g., terpene, flavonoid) with another, it also includes transforming one or more compound into a one or more different compound within the same class such as by any method known in the art. Exemplary methods, without limitation, include purification, racemization, enantiomeric inversion, isomerization, denaturization, sterilization, lyophilization, freeze-drying, homogenization, sonication, emulsification, gravimetric separation, aeration, gas infusion or shear force manipulation.

In some embodiments, the above method is repeated sequentially more than one, include 2, 3, 4, 5, or even more times to create a matrix including an entourage-effect from the sequential administration. As used herein, the “entourage-effect” refers to the residual effect of one or more compounds in the sequentially administered compositions.

### Definitions

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, particularly suitable methods and materials are described below.

When introducing elements of the embodiments described herein, the articles “a,” “an,” “the,” and “said” are intended to mean that there are one or more of the elements. The terms “comprising,” “including,” and “having” are intended to be inclusive and mean that there may be additional elements other than the listed elements.

As used herein, the following definitions shall apply unless otherwise indicated. For purposes of this disclosure, the chemical elements are identified in accordance with the Periodic Table of the Elements, CAS version, and the Handbook of Chemistry and Physics, 75th Ed. 1994. Additionally, general principles of organic chemistry are described in “Organic Chemistry,” Thomas Sorrell, University Science Books, Sausalito: 1999, and “March's Advanced Organic Chemistry,” 5th Ed., Smith, M. B. and March, J., eds. John Wiley & Sons, New York: 2001, the entire contents of which are hereby incorporated by reference to the extent they are consistent herewith.

The term “excipient” herein means any substance, not itself a therapeutic agent, used as a carrier or vehicle for delivery of a therapeutic agent to a subject or combined with a therapeutic agent (e.g., to create a pharmaceutical composition) to improve its handling or storage properties or to permit or facilitate formation of a dose unit of the composition.

The term “treat,” “treatment,” or “treating,” as used herein, means reducing or eliminating one or more clinical symptoms of insomnia, reducing the severity of one or more clinical symptoms of insomnia, or suppressing one or more clinical symptoms of insomnia.

The term “prodrug,” as used herein, means a pharmacological compound that is in an inactive form when administered, but is metabolized in vivo into an active form of the compound. Prodrugs are generally used to improve bioavailability in oral dosage forms, as well as selectivity of a desired target.

The term “emulsifying agent,” as used herein, refers to an agent capable of lowering surface tension between a non-polar and polar phase and includes compounds defined elsewhere as “self emulsifying” agents.

The abbreviation “CBDA,” as used herein, means cannabidiolic acid. The abbreviation “CBD,” as used herein, means cannabidiol. The abbreviation “CBN,” as used herein, means cannabinol. The abbreviation “CBGA,” as used herein, means cannabigerolic acid. The abbreviation “CBG,” as used herein, means cannabinoid. The abbreviation “CBCA,” as used herein, means cannabichromenic acid. The abbreviation “CBC,” as used herein, means cannabichromene. The abbreviation “CBDVA,” as used herein, means cannabidivarin acid. The abbreviation “CBDV,” as used herein, means cannabidivarin. The abbreviation “CBGVA,” as used herein, means cannabigerovarin acid. The abbreviation “THCA,” as used herein, means tetrahydrocannabinolic acid. The abbreviation “THC,” as used herein, means tetrahydrocannabinol. The abbreviation “THCVA,” as used herein, means tetrahydrocannabivarin carboxylic acid. The abbreviation “THCV,” as used herein, means tetrahydrocannabivarin.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Prophetic Example 1. Formulations

The cannabinoids in the delivery formulation dose include a combination of 9-A-tetrahrydrocannabinol (THC), cannabinol (CBN), cannabidiol (CBD), cannabichromene (CBC), and cannabigerol (CBG) with amounts outlined in Table 1.

TABLE 1
Composition of Cannabinoids in the delivery formulation dose.

| Compound | Amount (mg) |
|---|---|
| THC | 8-12 |
| CBN | 2-5 |
| CBD | 1-3, |
| | delayed release |
| CBC | <1 |
| CBG | <1 |

THC and CBN are used to promote somnolence and decrease sleep latency where the average time to maximum serum concentration (Tmax) is 0.5-2 hours post-administration of the delivery formulation. A low, delayed dose of CBD is used to promote awakening and minimize morning drowsiness/fatigue, where the Tmax of CBD is 6-8 hours postadministration of the delivery formulation.

The terpene composition in the formulation before loading into a delivery device or delivery formulation is outlined in Table 2.

TABLE 2
Composition of terpenes in the formulation before loading
into a delivery device or delivery formulation.

| Compound | Amount range (wt. %) |
|---|---|
| Borneol | 0.1-1 |
| Myrcene | 0.1-1 |
| Phytol | 0.1-1 |
| Terpinolene | 0.1-1 |
| beta-caryophyllene | 0.05-0.5 |
| Linalool | 0.05-0.5 |
| alpha-pinene | 0.01-1 |
| Camphene | 0.01-1 |
| Limonene | 0.01-1 |
| Humulene | 0.01-1 |
| Nerolidol | 0.01-1 |
| 1,8-cineole | <0.01 |
| Pulegone | <0.01 |

Borneol, mycene, terpenolene, beta-caryophyllene, and linalool are present in higher proportions to enhance blood-brain-barrier delivery of cannabinoids, activation of (gamma-Aminobutyric acid) GABAA receptors and/or sedation. alpha-pinene, camphene, limonene, humulene, and nerolidol are present in moderate proportions to enhance activation of GABAA and/or adenosine receptors, reduce anxiety, and/or minimize acetylcholinesterase (AchE) inhibition. 1,8-cineole, and pulegone are present in lower proportions to minimize AchE inhibition and/or prevent alertness.

The flavonoid composition in the formulation before loading into a delivery device is outlined in Table 3.

TABLE 3
Compositions of flavonoids in the formulation before loading
into a delivery device or delivery formulation.

| | Amount Range |
|---|---|
| Compound | (% by weight) |
| β-sitosterol | 0.1-1 |
| cannaflavin A | 0.1-1 |
| luteolin | 0.05-0.5 |
| orientin | 0.05-0.5 |
| apigenin | Less than 0.01 |
| kaempferol | Less than 0.01 |
| quercetin | Less than 0.01 |

Beta-sitosterol and cannaflavin A are present in higher proportions to reduce alertness. Luteolin and orientin are present in moderate proportions to enhance GABAA activation and/or minimize AchE inhibition. Apigenin, kaempferol, and quercetin are present in lower proportions to minimize adenosine receptor antagonism, and/or minimize AchE inhibition.

The composition of cannabinoids, terpenes, and flavonoids outlined in Table 1, Table 2, and Table 3 are combined into a final formulation comprising of excipient(s), polymer(s), vehicle(s), and/or flavorants to modify adsorption, solubility, viscosity, physical structure, and/or tastes.

In an exemplary embodiment, the composition may include about 10 mg THC, about 1-2 mg of CBD (extended release), about 2-3 mg CBN, about 1 mg CBG, and about 1 mg CBC, about 0.1% (or 0.1-1%) by weight of: borneol, myrcene, phytol and, terpinolene; about 0.05% (or 0.05-0.5%) by weight of: beta-caryophyllene, and linalool; about 0.01% (or 0.01-0.1%) by weight of: alpha-pinene, camphene, limonene, humulene, and nerolidol; about 0.01% by weight of: 1,8-cineole and, pulegone. Optionally, the composition may further include about 0.01% of luteolin, about 0.01% kaempferol, about 0.01% quercetin, about 0.01% apigenin, about 0.01-0.1% beta-sitosterol, about 0.1-1% cannaflavin A, and about 0.05-1% orientin.

In another exemplary embodiment, the composition may comprise about 10 mg THC, about 10 mg of CBD (gradual release), about 2-3 mg CBN, about 1 mg CBG, about 1 mg CBC, about 0.1% (or 0.1-1%) by weight of: borneol, myrcene, alpha-pinene and, limonene about 0.05% (or 0.05-0.5%) by weight of: linalool about 0.01% (or 0.01-0.1%) by weight of: terpinolene, beta-caryophyllene, pulegone, phytol, humulene, 1,8-cineole, and nerolidol, about 0.01% by weight of: camphene, about 0.01% of luteolin, about 0.01% kaempferol, about 0.01% quercetin, about 0.01% apigenin, about 0.01-0.1% beta-sitosterol, about 0.1-1% cannaflavin A, and about 0.05-1% orientin.

### Example 2. Investigation of Cannabis for the Treatment of Insomnia Symptoms

The primary objective of this Example was to evaluate the efficacy of cannabis oils on sleep in insomnia participants using measurements of Insomnia Severity Index (ISI), actigraphy, and sleep diary. ISI is a reliable and valid instrument used to quantify perceived insomnia severity (Morin et al., 2011, the disclosure of which is hereby incorporated by reference in its entirety). A score of <8 on the ISI implies no clinical insomnia, 8-14 implies subthreshold insomnia, 15-21 implies clinical insomnia (mild severity), and 22-28 implies severe clinical insomnia. Actigraphy is a non-invasive method of monitoring activity/rest cycles by measuring gross motor activity, and can be used as a proxy to measure sleep parameters. Participants wore a Motionlogger Micro Watch from Ambulatory Monitoring, Inc. (AMI) for the duration of the study. The Motionlogger devices were set to 1-minute epoch lengths and AMI's companion Action-W2 software was used to acquire total time asleep, latency to sleep, longest time awake after sleep onset, total time awake, and sleep efficiency. Sleep diaries were completed daily, with self-reported scales of -rested after sleep, quality of sleep, sleep latency, and sleep duration.

The secondary objective was to measure the effectiveness of cannabis oils on quality of life in insomnia participants. Quality of life was assessed by EQ-5D-a clinically-validated questionnaire that produces a standard, single index value as a measurement of health status (van Reenen and Janssen, 2015, the disclosure of which is hereby incorporated by reference in its entirety).

### Participants

Participants who were experiencing chronic insomnia, held a medical document to possess medical cannabis in accordance with Health Canada's Medical Marijuana Access Regulations (ACMPR) and were planning on using medical cannabis were recruited. Eligible participants were adult men and non-pregnant women aged 25-75 who had an Insomnia Severity Index (ISI)≥10 and self-reported sleep difficulty for the past month. Participants were excluded if they used medical cannabis in the past 3 months, used cannabis regularly (>2 times/week) in the past year, used cannabis in the past week, self-reported other sleep disorders, and/or had 3 or more comorbid disorders.

Participants were advised to report any adverse and severe adverse events to the clinic's team and appropriate regulatory bodies. The average level of dizziness was increased at each chemovar level, correlating with an increase in THC content.

### Methods

This observational, open-label Example investigating the relationship between cannabis and sleep was conducted at a clinic. The 6-week long study was segmented into three distinct stages. Each stage signified a specific THC:CBD concentration. Participants attended scheduled visits with the clinical team (baseline, weeks 2, 4, and 6) to complete questionnaires, change medical cannabis stage, and obtain their cannabis oil. Telephone interviews were also conducted (weeks 1, 4, and 5) to complete weekly questionnaires.

Three stages of cannabis oil chemovar types (high CBD, 1:1 THC:CBD, and high THC) were provided to participants over a period of 2-weeks per chemovar type consisting of a 1-week escalating dose phase followed by a 1-week maintenance dose phase. Within the chemovar types, a total of 9 different preparations of medical cannabis oils were available to participants. Table 4 outlines the CBD and/or THC concentration ranges, and volumes of the 9 medical cannabis preparations within the 3 chemovar types.

TABLE 4
Concentrations of THC and CBD in Study Oils.

| | THC | CBD | | |
|---|---|---|---|---|
| | concentration | concentration | | Volume of |
| Chemovar | (mg/mL) | (mg/mL) | Week | oil (mL) |
| High CBD | <0.7-1 | 10-20 | 1 | 0.25-0.5 |
| | | | 2 | 1.0 |
| 1:1 THC:CBD | 5-10 | 8-15 | 3 | 0.25-0.5 |
| | | | 4 | 1.0 |
| High THC | 14-26.3 | <0.7-1 | 5 | 0.5-0.75 |
| | | | 6 | 1.0 |

All participants were given a daily dose 30-60 minutes before bed in the form of an oral liquid, tablet, lipid matrix sublingual tablet, or lipid matrix sublingual spray.

The lot-specific cannabinoid, terpene, and flavonoid profiles used for this study are listed in Table 5.

TABLE 5
Cannabinoid, Terpene, and Flavonoid Concentration in Study Oils.

| | Oil 1 | Oil 2 | Oil 3 | Oil 4 | Oil 5 | Oil 6 | Oil 7 | Oil 8 | Oil 9 |
|---|---|---|---|---|---|---|---|---|---|
| Chemovars | (mg/mL) | (mg/mL) | (mg/mL) | (mg/mL) | (mg/mL) | (mg/mL) | (mg/mL) | (mg/mL) | (mg/mL) |
| Cannabinoids: | | | | | | | | | |
| THCA | — | — | 0.04 | 0.01 | 0.09 | 0.61 | 0.21 | 0.08 | 0.07 |
| THC | 0.38 | 0.76 | 4.4 | 6.0 | 9.73 | 14.9 | 16 | 23.2 | 24.2 |
| CBDA | 0.29 | 0.2 | 0.38 | 0.41 | 0.17 | 0.16 | — | — | — |
| CBD | 9.02 | 19 | 6.29 | 8.0 | 13.8 | 0.66 | 0.13 | 0.14 | 0.08 |
| CBN | — | — | 0.2 | 0.23 | 0.26 | 0.34 | 0.59 | 0.24 | 0.44 |
| CBGA | 0.04 | 0.08 | 0.03 | 0.07 | 0.07 | 0.15 | 0.03 | 0.03 | 0.03 |
| CBG | 0.18 | 0.2 | 0.18 | 0.21 | 0.44 | 0.91 | 0.49 | 0.61 | 0.7 |
| CBCA | 0.003 | 0.0035 | 0.01 | 0.003 | 0.0015 | 0.003 | 0.003 | 0.003 | 0.004 |
| CBC | 0.47 | 0.84 | 0.36 | 0.59 | 0.69 | 0.3 | 0.27 | 0.64 | 0.41 |
| THCVA | 0.01 | 0.02 | 0.01 | 0.01 | 0.01 | 0.01 | 0.01 | 0.01 | 0.01 |
| THCV | 0.01 | 0.03 | 0.05 | 0.08 | 0.11 | 0.14 | 0.18 | 0.2 | 0.17 |
| CBDVA | 0.02 | 0.02 | 0.04 | 0.04 | 0.03 | 0.06 | 0.1 | 0.03 | 0.08 |
| CBDV | 0.02 | 0.06 | 0.05 | 0.05 | 0.06 | 0.01 | 0.03 | — | 0.04 |
| CBGVA | — | — | — | — | — | — | — | — | — |
| Terpenes: | | | | | | | | | |
| alpha-Pinene | 0.02 | — | — | — | — | 0.05 | — | — | — |
| Camphene | — | — | — | — | — | — | — | — | — |
| Sabinene | — | — | — | — | — | — | — | — | — |
| beta-Pinene | 0.01 | — | — | — | — | 0.02 | — | — | — |
| beta-Myrcene | 0.11 | 0.008 | — | 0.01 | 0.01 | 0.42 | 0.01 | 0.01 | — |
| p-Mentha-1,5- | — | — | — | — | — | — | — | — | — |
| diene | | | | | | | | | |
| (+)-3-Carene | — | — | — | — | — | — | — | — | — |
| alpha-Terpinene | — | — | — | 0.01 | — | — | — | — | — |
| Limonene | 0.03 | — | — | 0.02 | — | 0.03 | 0.02 | — | — |
| Eucalyptol | — | — | — | — | — | — | — | — | — |
| trans-beta- | — | — | — | — | — | — | — | — | — |
| Ocimeme | | | | | | | | | |
| Beta-Ocimene | — | — | — | — | — | 0.05 | — | — | — |
| Gamma- | — | — | — | — | — | — | — | — | — |
| Terpinene | | | | | | | | | |
| Sabiene Hydrate | — | — | — | — | — | — | — | — | — |
| Fenchone | — | — | — | — | — | — | — | — | — |
| Isomers | | | | | | | | | |
| Terpinolene | — | — | — | 0.02 | — | 0.01 | — | — | — |
| Linalool | 0.01 | — | — | — | 0.01 | 0.03 | — | — | 0.01 |
| Fenchyl Alcohol | 0.006 | — | 0.004 | — | 0.003 | — | — | — | 0.01 |
| Camphor Isomers | — | — | — | — | — | — | — | — | — |
| Isopulegol | — | — | — | — | — | — | — | — | — |
| Isoborneol | — | — | — | — | — | — | — | — | — |
| Borneol Isomers | 0.02 | 0.02 | 0.02 | 0.02 | 0.02 | — | 0.02 | 0.02 | 0.02 |
| Hexahydrothymol | — | — | — | — | — | — | — | — | — |
| Alpha-Terpineol | — | — | — | — | 0.02 | 0.01 | — | — | 0.03 |
| Ganna-Terpineol | — | — | — | — | — | — | — | — | — |
| Gamma- | — | — | — | — | — | — | — | — | — |
| Terpineol | | | | | | | | | |
| Geranyl Acetate | — | — | — | — | — | — | — | — | — |
| Pulegone | — | — | — | — | — | — | — | — | — |
| Nerol | — | — | — | — | — | — | — | — | — |
| Alpha-Cedrene | — | — | — | — | — | — | — | — | — |
| Trans- | 0.02 | 0.03 | 0.03 | 0.05 | 0.1 | 0.1 | 0.03 | 0.07 | 0.11 |
| caryophyllene | | | | | | | | | |
| Alpha-humulene | 0.01 | 0.02 | 0.02 | 0.03 | 0.04 | 0.04 | 0.02 | 0.03 | 0.06 |
| Valencene | — | — | — | — | — | — | — | — | — |
| Cis-nerolidol | — | — | — | — | — | — | — | — | — |
| Trans-nerolidol | — | — | — | 0.01 | 0.05 | — | — | 0.05 | 0.03 |
| Caryophyllene | — | — | — | — | — | — | — | — | — |
| oxide | | | | | | | | | |
| Guaiol | 0.03 | 0.09 | 0.05 | — | 0.14 | 0.07 | 0.07 | — | 0.07 |
| Cedrol | — | — | — | — | — | — | — | — | — |
| Alpha-Bisabolol | 0.03 | 0.2 | 0.03 | — | 0.36 | 0.11 | 0.03 | 0.03 | 0.13 |
| Flavonoids | | | | | | | | | |
| Quercitin | — | — | — | — | — | — | — | — | — |
| Apigenin-7-O- | — | — | — | — | — | — | — | — | — |
| glucoside | | | | | | | | | |
| Luteolin | — | — | — | — | — | — | — | — | — |
| Apigenin | — | — | — | — | — | — | — | — | — |
| Kaempferol | — | — | — | — | — | — | — | — | — |
| Cannflavin B | 0.003 | 0.015 | 0.001 | 0.0003 | 0.003 | 0.002 | 0.002 | 0.0002 | 0.001 |
| Cannflavin A | 0.003 | 0.018 | — | — | 0.012 | 0.007 | 0.009 | 0.001 | 0.003 |
| Myricetin | — | — | — | — | — | — | — | — | — |
| Luteolin-7-O- | — | — | — | — | — | — | — | — | — |
| glucoside | | | | | | | | | |

* “—” indicated below detection limit

The experimental drug was provided in UV-protected bottles and supplied with a 1 mL syringe for precise dosing.

In order to assess the safety of cannabis, occurrences of all adverse events and serious adverse events were recorded. Tolerability was assessed by participants feelings and tolerability of “high.”

The mean differences of each measured parameter were compared at each week from each medical cannabis oil to baseline using paired t-tests with a 95% confidence interval. RESULTS

58 participants were enrolled in the study. A total of 40 participants (13 males and 27 females) completed the 6-week long study. Participants had the ability to drop out of the study at any time. 16 participants withdrew from the study and 2 were lost to follow-up.

There were statistically significant decreases in ISI values for each oil compared to baseline. There were clinically significant differences in ISI values from weeks 3 to 6 (Oils 3-9) compared to baseline.

There was a statistically significant increase in total time asleep between oil 4 and baseline. There were no statistically significant differences in actigraphy measures (latency to sleep, longest time awake after sleep onset, total time awake, and sleep efficiency) between any of the 9 cannabis oils and baseline.

There was a statistically significant improvement in perceived: ‘rested after sleep’ with oils 5 and 9 compared to baseline, ‘sleep quality’ with oils 2 and 3 compared to baseline, and reduction in ‘latency to sleep’ with oils 6 and 9.

EQ-5D data was statistically improved for oils 1, 2, 3, 5, 6, and 8 when compared to baseline. Dose range had seemingly little impact in this study on EQ-5D data. Nonetheless, these results indicate that cannabis oils bettered participants self-reported health status.

No serious adverse events were reported.

### REFERENCES

Morin C M, Belleville G, Bélanger L, Ivers H. 2011. The Insomnia Severity Index: Psychometric indicators to detect insomnia cases and evaluate treatment response. Sleep: 34 (5): 601-8.
van Reenen M, Janssen B. 2015. Version 2.1: EQ-5D-5L User Guide: Basic information on how to use the 5Q-5D-5L instrument. EuroQol Research Foundation. The Netherlands.

All cited references are herein expressly incorporated by reference in their entirety to the extent they are consistent herewith.

Whereas particular embodiments have been described above for purposes of illustration, it will be appreciated by those skilled in the art that numerous variations of the details may be made without departing from the disclosure as described in the appended claims.

1. A composition comprising:
a cannabidiol (CBD), derivative, intermediate, or prodrug thereof, and combinations thereof in an amount of from about 0.5 mg/mL to about 25 mg/mL;
a tetrahydrocannabinol (THC), derivative, or intermediate thereof, and combinations thereof in an amount of from about 0.5 mg/mL to about 30 mg/mL;
at least one terpene; and
at least one flavonoid.

2. The composition of claim 1, wherein the ratio of the cannabidiol (CBD), derivative, intermediate, or prodrug thereof, and combinations thereof: the tetrahydrocannabinol (THC), derivative, or intermediate thereof, and combinations thereof is 1:1.

3. The composition of claim 1, wherein the cannabidiol (CBD), derivative, and intermediate, or prodrug thereof is selected from the group consisting of CBDA, CBD, CBN, CBGA, CBG, CBCA, CBC, CBDVA, CBDV, CBGVA and combinations thereof.

4. The composition of claim 1, wherein the cannabidiol (CBD), derivative, and intermediate, or prodrug thereof is cannabidiol (CBD).

5. The composition of claim 1, wherein the cannabidiol (CBD) is present in the composition from about 8 mg/mL to about 15 mg/mL.

6. The composition of claim 1, wherein the tetrahydrocannabinol (THC), derivative, or intermediate thereof is selected from the group consisting of THCA, THC, THCVA, THCV, and combinations thereof.

7. The composition of claim 1, wherein the tetrahydrocannabinol (THC), derivative, or intermediate thereof is tetrahydrocannabinol (THC).

8. The composition of claim 1, wherein the tetrahydrocannabinol (THC) is present in the composition from about 5 mg/mL to about 10 mg/mL.

9. The composition of claim 1, wherein the at least one terpene is selected from the group consisting of alpha-Pinene, beta-Pinene, beta-Myrcene, alpha-Terpinene, Limonene, beta-Ocimene, Terpinolene, Linalool, Fenchyl Alcohol, Borneol Isomers, Alpha-Terpineol, Trans-caryophyllene, Alpha-humulene, Trans-nerolidol, Guaiol, Alpha-Bisabolol, and combinations thereof.

10. The composition of claim 1, wherein the at least one terpene is present in the composition from about 0.1 mg/mL to about 1 mg/mL.

11. The composition of claim 1, wherein the at least one flavonoid is selected from the group consisting of quercetin, apigenin-7-O-glucoside, luteolin, apigenin, kaempferol, cannflavin B, cannflavin A, myricetin, luteolin-7-O-glucoside, and combinations thereof.

12. The composition of claim 1, wherein the at least one flavonoid is present in the composition from about 0.0001 mg/mL to about 0.01 mg/mL.

13. The composition of claim 1, further comprising a pharmaceutical acceptable excipient.

14. The composition of claim 1, wherein the composition is formulated into a lipid matrix.

15. A method of treating insomnia in a subject in need thereof, the method comprising: administering to the subject a therapeutically effective amount of a composition comprising: a cannabidiol (CBD), derivative, intermediate, or prodrug thereof, and combinations thereof in an amount of from about 0.5 mg/mL to about 25 mg/mL; a tetrahydrocannabinol (THC), derivative, or intermediate thereof, and combinations thereof in an amount of from about 0.5 mg/mL to about 30 mg/mL; at least one terpene; and at least one flavonoid.

16. The method of claim 15, wherein the ratio of the cannabidiol (CBD), derivative, intermediate, or prodrug thereof, and combinations thereof: the tetrahydrocannabinol (THC), derivative, or intermediate thereof, and combinations thereof is 1:1.

17. The method of claim 15, wherein the cannabidiol (CBD), derivative, and intermediate, or prodrug thereof is selected from the group consisting of CBDA, CBD, CBN, CBGA, CBG, CBCA, CBC, CBDVA, CBDV, CBGVA and combinations thereof.

18. The method of claim 15, wherein the tetrahydrocannabinol (THC), derivative, or intermediate thereof is selected from the group consisting of THCA, THC, THCVA, THCV, and combinations thereof.

19. The method of claim 15, wherein the at least one terpene is selected from the group consisting of alpha-Pinene, beta-Pinene, beta-Myrcene, alpha-Terpinene, Limonene, beta-Ocimene, Terpinolene, Linalool, Fenchyl Alcohol, Borneol Isomers, Alpha-Terpineol, Trans-caryophyllene, Alpha-humulene, Trans-nerolidol, Guaiol, Alpha-Bisabolol, and combinations thereof.

20. The method of claim 15, wherein the at least one flavonoid is selected from the group consisting of quercetin, apigenin-7-O-glucoside, luteolin, apigenin, kaempferol, cannflavin B, cannflavin A, myricetin, luteolin-7-O-glucoside, and combinations thereof.

21.-24. (canceled)
